SEQUENCE LISTING (4 sequences)

SEQ ID NO: 1 - protein, 60 aa, Escherichia coli
LAYGSQGDLNPLINEISKIISAAGSFDVKEERTAASLLQLSGNASDFSYGRNSITLTTSA

SEQ ID NO: 2 - protein, 60 aa, synthetic construct
LAYGSQGDLNPLINEISKIISAAGNFDVKEERTAASLLQLSGNASDFSYGRNSITLTTSA

SEQ ID NO: 3 - protein, 163 aa, Ralstonia eutropha
MILTPEQVAAAQKANLETLFGLTTKAFEGVEKLVELNLQVVKTSFAEGVDNAKKALSAKDAQELLAIQAAAVQPVAEKTLAYTRHLYEIASETQSEFTKVAEAQLAEGSKNVQALVENLAKNAPAGSESTVAIVKSAISAANNAYESVQKATKQAVEIAETNF

SEQ ID NO: 4 - protein, 185 aa, Pseudomonas sp. B1 4-6
MSFFNSEKLQTAQKANLDLLQQISGKVFASVEQLSQLQFKALRDSTEEHFEGVRKLLAVRDPQGFADLQASFTQPSAQAERLAEFNRQVQALIVGTQSDIAKLTSSQVEAGTQQVNEFVESISKNAPAGSEPVVAAFKSGLANASTVFESAQKAVKQASDVAQSNFDAATKAAPEASAKATSGNK

(19) United States
(12) Patent Application Publication
(10) Pub. No.: US 2025/0019729 A1
(43) Pub. Date: Jan. 16, 2025

(54) IN PHA PRODUCTION

(71) Applicant: ExxonMobil Technology and Engineering Company, Baytown, TX (US)

(72) Inventor: Leticia R. CARRILLO-MAX, High Bridge, NJ (US)

(21) Appl. No.: 18/658,087

(22) Filed: May 8, 2024

Related U.S. Application Data
(60) Provisional application No. 63/512,956, filed on Jul. 11, 2023.

(51) Int. Cl. C12P 7/625; C07K 14/195; C07K 14/245; C12N 15/70

(52) U.S. Cl. CPC C12P 7/625; C07K 14/195; C07K 14/245; C12N 15/70

(57) ABSTRACT

An embodiment of the present disclosure provides an isolated nucleic acid molecule comprising a sequence encoding a protein having a PhaP1 domain and a HlyA domain, wherein the HlyA domain differs from the wild-type HlyA domain of E. coli (SEQ ID NO.: 1)

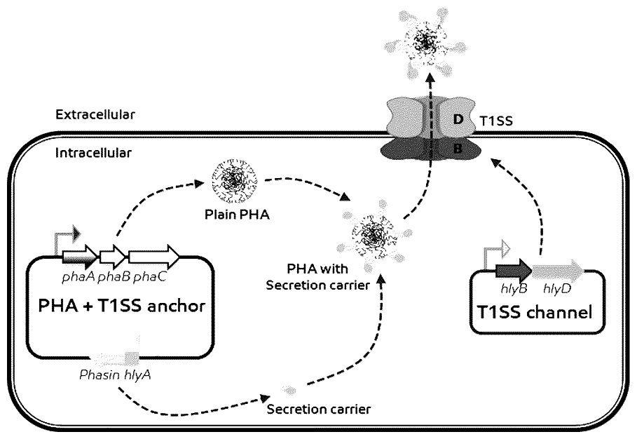

SEQ ID NO. 1:
LAYGSQGDLNPLINEISKIISAAGSFDVKEERTAAS
LLQLSGNASDFSYGRNSITLTTSA

in at least the wild-type amino acid residue S (serine) at position 25 (underlined above) is replaced with the amino acid residue N (asparagine).

### CROSS-REFERENCE TO RELATED APPLICATION

This application claims the benefit of U.S. Provisional Application 63/512,956, filed Jul. 11, 2023, entitled “Improvement in PHA Production”, the entirety of which is incorporated by reference.

### REFERENCE TO A SEQUENCE LISTING

This application contains references to peptide sequences which have been submitted electronically via EFS-Web as an XML formatted sequence listing with a file name “PM2022049 sequence listing.xml”, with a creation date of Jun. 25, 2024, and a size of 5 KB, as a sequence listing file. The aforementioned sequence listing submitted via EFS-WEB is part of the specification and is hereby incorporated by reference in its entirety.

### FIELD

The present disclosure relates to improvements in the production of polyhydroxyalkanoates (PHAs) and to related genes, vectors, expression cassettes, organisms and methods useful in the production of PHAs.

### BACKGROUND

Polyhydroxyalkanoates (PHAs) may be produced by numerous microorganisms through fermentation of sugars or lipids, which are derivable from various sources and feedstocks, and in nature they serve as a carbon boand energy store within the organism.

PHAs are thermoplastic with melting points from 40° C. to 180° C. PHAs typically have good UV-stability and low permeability to water.

PHAs have great potential to both replace and complement other plastics materials including petrochemical-derived plastics.

PHAs have been widely used in the medical device industry, for example, as sutures, surgical mesh, orthopaedic pins, stents, dressing and scaffolds. Subject to improvements in the economics of commercial production, there is potential for PHAs to be used more widely.

PHAs are synthesized by many bacteria as intracellular inclusion bodies which are enclosed by a double layer cell membrane. The enclosure in a cell membrane contributes significantly to high costs of downstream processing which may contribute to as much as half of the total material cost. Current methods of downstream processing involve the use of considerable quantities of relatively expensive chemicals and solvents such as hypochlorite and chloroform and/or energy-intensive processes of extraction and polymer precipitation. The costs and complexities arising from extraction and precipitation limits industrial-scale production of PHAs. There have been attempts to overcome the extraction problem including engineering organisms producing PHAs with a lytic or phage mechanism for regulated release and ease of extraction. However, the rates of extraction have not always been as high as hoped for (Martinez et al., 2011; June et al. 2005; Leong et al., 2018). An alternative approach to the extraction problem is to engineer the organism to secrete PHA. Rahman et al. (2013) developed a secretory method for a specific form of PHA, polyhydroxybutyrate (PHB). U.S. Pat. No. 9,169,300 to Miller et al discloses chimeric polypeptides used to transport PHA extracellularly.

Because PHB, and PHAs in general, are non-proteinaceous polymers they cannot be directly targeted for secretion. The method of Rahman et al. targets a phasin protein with the ability to bind to PHA and thereby surround PHA granules. PHA granules surrounded by phasin are exported from the cell via a type 1 secretion (T1SS) pathway. The success of the method developed by Rahman et al. was limited, with only 36% of total PHB produced located in the secreted fraction and 64% of the PHB retained in the cellular fraction. There is a need to improve the method of Rahman et al. and also Miller et al. Such improvements would desirably result in a far higher proportion of PHA being secreted with little or none remaining in the cellular fraction, thus allowing a step of lysing and extracting the PHA from the cell to be reduced or preferably eliminated which would increase efficiency and lower the cost of PHA production as well as bringing other technical advantages including applicability to a continuous or semi-continuous process and improvements in the quality of PHA and products made therefrom by removing the use of extraction methods which have potential to degrade quality. There is also a need to provide further technical advantages to PHA production by the method of Rahman et al. including the selective production of specific copolymers with advantageous thermophysical properties and the ability of the organism to utilise efficiently advantageous feedstocks, including lignocellulose by-products.

### The PHA Production Pathway

Ralstonia eutropha (also known as Cupriavrdus necator) may be viewed as an archetypal microbial organism for the production of PHAs. Ralstonia eutropha contains an operon (known as the phaCAB operon) of three genes; PHA synthase (phaC), O-ketothiolase (phaA) and acetoacetyl-CoA reductase (phaB).

As illustrative of PHA production in general, the production of PHB may be carried out via a 3-step enzymatic reaction. Firstly, two acetyl-CoA molecules are converted into acetoacetyl-CoA by a condensation reaction catalysed by β-ketothiolase (phaA), then a stereoselective reduction takes place to (R)-3-hydroxybutryl-CoA by the NADPH-dependent acetoacetyl-CoA reductase (phaB) and finally the monomer of PHB is polymerized by PHB synthase (phaC).

PHA molecules in nature are coated with the PHA phasin protein (PhaP1). Rahman et al. engineered the PhaP1 protein to add a HlyA (hemolysin A) signal peptide sequence which targeted it for section with the type 1 secretion machinery.

The present invention is based in part on site-directed modifications of the PhaP1 and HlyA sequences to increase export efficiency.

### SUMMARY

In its first aspect, an embodiment of the present disclosure provides an isolated nucleic acid molecule comprising a sequence encoding a protein having a PhaP1 domain and a HlyA domain, wherein the HlyA domain differs from the wild-type HlyA domain of E. coli (SEQ ID.: 1)

SEQ ID NO. 1:
LAYGSQGDLNPLINEISKIISAAGSFDVKEERTAAS
LLQLSGNASDFSYGRNSITLTTSA

in at least the wild-type amino acid residue S (serine) at position 25 (underlined above) is replaced with the amino acid residue N (asparagine).

In a second aspect, an embodiment of the present disclosure provides a plasmid comprising an isolated nucleic acid molecule of the first aspect of the disclosure.

In a third aspect, an embodiment of the present disclosure provides an expression cassette comprising an isolated nucleic acid molecule of the first aspect of the disclosure, and a promoter to which it is operably linked.

In a fourth aspect, an embodiment of the present disclosure provides a vector comprising an expression cassette of the third aspect.

In a fifth aspect, an embodiment of the present disclosure provides a prokaryotic cell comprising a plasmid of the second aspect of the disclosure or an expression cassette of the third aspect of the disclosure, or a vector of the fourth aspect of the disclosure.

In a sixth aspect, an embodiment of the present disclosure provides a culture comprising prokaryotic cells of the fifth aspect.

In a seventh aspect, an embodiment of the present disclosure provides a method of obtaining PHA comprising culturing bacterial cells of the fifth aspect for sufficient time and under suitable conditions for said cells to produce PHA and secrete it into the extracellular medium, and separating PHA from the extracellular medium.

In an eighth aspect, an embodiment of the present disclosure provides a method for transforming a prokaryotic cell, the method comprising:
introducing a vector of the fourth aspect of the disclosure into the prokaryotic cell and selecting for a transformed prokaryotic cell.

A ninth aspect of an embodiment of the present disclosure provides an isolated nucleic acid molecule comprising a sequence encoding a protein having a PhaP1 domain and a HlyA domain wherein the PhaP1 domain differs from the wild-type Ralstonia eutropha sequence (SEQ ID NO:3)

SEQ ID NO: 3:
MILTPEQVAAAQKANLETLFGLTTKAFEGVEKLVELNLQVVKTSF
AEGVDNAKKALSAKDAQELLAIQAAAVQPVAEKTLAYTRHLYEIA
SETQSEFTKVAEAQLAEGSKNVQALVENLAKNAPAGSESTVAIVK
SAISAANNAYESVQKATKQAVEIAETNF

in one or more of the following respects:
the presence of amino acid residue N at position 24
the presence of amino acid residues ATL at positions 43 to 45
the presence of amino acid residue N at position 48
the presence of amino acid residue E at position 50
the presence of amino acid residue L at position 67
the presence of amino acid residue L at position 71
the presence of amino acid residue V at position 79
the presence of amino acid residue S at position 83
the presence of amino acid residue A at position 95
the presence of amino acid residue I at position 105
the presence of amino acid residue D at position 117
the presence of amino acid residue V at position 119
the presence of amino acid residue A at position 130
the presence of amino acid residue L at position 133
the presence of amino acid residue V at position 138
the presence of amino acid residue S at position 161.

A tenth and eleventh aspect of embodiments of the present disclosure provide, respectively, a plasmid and an expression cassette comprising an isolated nucleic acid molecule according to the ninth aspect of the disclosure.

A twelfth aspect of an embodiment of the present disclosure provides a vector comprising an expression cassette of the eleventh aspect of the disclosure.

A thirteenth aspect of an embodiment of the present disclosure provides a prokaryotic cell comprising a plasmid of the tenth aspect of the disclosure or an expression cassette of the eleventh aspect of the disclosure or a vector of the twelfth aspect of the disclosure.

A fourteenth aspect of an embodiment of the present disclosure provides a culture comprising prokaryotic cells of the thirteenth aspect of the disclosure.

A fifteenth aspect of an embodiment of the present disclosure provides a method of obtaining PHA comprising culturing prokaryotic cells of the thirteenth aspect for sufficient time and under suitable conditions for said cells to produce PHA and secrete it into the extracellular medium and separating PHA from the extracellular medium.

A sixteenth aspect of an embodiment of the present disclosure provides a method for transforming a prokaryotic cell, the method comprising: introducing a vector of the twelfth aspect of the disclosure into the prokaryotic cell and selecting for a transformed prokaryotic cell.

### DESCRIPTION OF THE DRAWINGS

The FIGURE is a diagrammatic representation of a bacterial cell producing PHAs and secreting them via the T1SS machinery.

### DETAILED DESCRIPTION

In its first aspect, an embodiment of the present disclosure provides an isolated nucleic acid molecule comprising a sequence encoding a protein having a PhaP1 domain and a HlyA domain, wherein the HlyA domain differs from the wild-type HlyA domain of E. coli (SEQ ID.: 1)

SEQ ID NO. 1:
LAYGSQGDLNPLINEISKIISAAGSFDVKEERTAAS
LLQLSGNASDFSYGRNSITLTTSA

in at least the wild-type amino acid residue S (serine) at position 25 (underlined above) is replaced with the amino acid residue N (asparagine).

In a second aspect, an embodiment of the present disclosure provides a plasmid comprising an isolated nucleic acid molecule of the first aspect of the disclosure.

In a third aspect, the present disclosure provides an expression cassette comprising an isolated nucleic acid molecule of the first aspect of the disclosure, and a promoter to which it is operably linked.

In a fourth aspect, the present disclosure provides a vector comprising an expression cassette of the third aspect.

In a fifth aspect, the present disclosure provides a prokaryotic cell comprising a plasmid of the second aspect of the present disclosure or an expression cassette of the third aspect of the present disclosure, or a vector of the fourth aspect of the present disclosure.

In a sixth aspect, the present disclosure provides a culture comprising prokaryotic cells of the fifth aspect.

In a seventh aspect, the present disclosure provides a method of obtaining PHA comprising culturing bacterial cells of the fifth aspect for sufficient time and under suitable conditions for said cells to produce PHA and secrete it into the extracellular medium, and separating PHA from the extracellular medium.

In an eighth aspect, the present disclosure provides a method for transforming a prokaryotic cell, the method comprising:

introducing a vector of the fourth aspect of the present disclosure into the prokaryotic cell and selecting for a transformed prokaryotic cell.

A ninth aspect of the present disclosure provides an isolated nucleic acid molecule comprising a sequence encoding a protein having a PhaP1 domain and a HlyA domain wherein the PhaP1 domain differs from the wild-type Ralstonia eutropha sequence (SEQ ID NO: 3):

SEQ ID NO: 3:
MILTPEQVAAAQKANLETLFGLTTKAFEGVEKLVELNLQVVKTSF
AEGVDNAKKALSAKDAQELLAIQAAAVQPVAEKTLAYTRHLYEIA
SETQSEFTKVAEAQLAEGSKNVQALVENLAKNAPAGSESTVAIVK
SAISAANNAYESVQKATKQAVEIAETNF

in one or more of the following respects:
the presence of amino acid residue N at position 24
the presence of amino acid residues ATL at positions 43 to 45
the presence of amino acid residue N at position 48
the presence of amino acid residue E at position 50
the presence of amino acid residue L at position 67
the presence of amino acid residue L at position 71
the presence of amino acid residue V at position 79
the presence of amino acid residue S at position 83
the presence of amino acid residue A at position 95
the presence of amino acid residue I at position 105
the presence of amino acid residue D at position 117
the presence of amino acid residue V at position 119
the presence of amino acid residue A at position 130
the presence of amino acid residue L at position 133
the presence of amino acid residue V at position 138
the presence of amino acid residue S at position 161.

A tenth and eleventh aspect of the present disclosure provide, respectively, a plasmid and an expression cassette comprising an isolated nucleic acid molecule according to the ninth aspect of the present disclosure.

A twelfth aspect of the present disclosure provides a vector comprising an expression cassette of the eleventh aspect of the present disclosure.

A thirteenth aspect of the present disclosure provides a prokaryotic cell comprising a plasmid of the tenth aspect of the present disclosure or an expression cassette of the eleventh aspect of the present disclosure or a vector of the twelfth aspect of the present disclosure.

A fourteenth aspect of the present disclosure provides a culture comprising prokaryotic cells of the thirteenth aspect of the present disclosure.

A fifteenth aspect of the present disclosure provides a method of obtaining PHA comprising culturing prokaryotic cells of the thirteenth aspect for sufficient time and under suitable conditions for said cells to produce PHA and secrete it into the extracellular medium and separating PHA from the extracellular medium.

A sixteenth aspect of the present disclosure provides a method for transforming a prokaryotic cell, the method comprising:
introducing a vector of the twelfth aspect of the present disclosure into the prokaryotic cell and selecting for a transformed prokaryotic cell.

### Definitions

Unless defined otherwise, al technical and scientific terms used herein have the same meaning as commonly understood by one of ordinary skill in the art to which this invention belongs. In case of conflict, the present application, including the definitions provided therein, will prevail.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

To facilitate an understanding of the present invention, a number of terms and phrases are defined below.

As used in the present disclosure and claims, the singular forms “a,” “an,” and “the” include plural forms unless the context clearly dictates otherwise.

Wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include “A and B”, “A or B”, “A”, and “B”.

The terms “cell” (e.g., host cell) and “cell culture” include the primary subject cells and any progeny thereof, without regard to the number of transfers. It should be understood that not all progeny are exactly identical to the parental cell (due to deliberate or inadvertent mutations or differences in environment). However, such altered progeny are included in these terms, so long as the progeny retain the same functionality as that of the originally transformed cell.

The term “gene” is used broadly to refer to any segment of nucleic acid molecule (typically DNA, but optionally RNA) that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions, 3′ untranslated regions, introns, etc. Further, genes may optionally comprise regulatory sequences required for their expression, and such sequences may be, e.g., sequences that are not transcribed or translated. Genes can be obtained from a variety of sources, including cloning from a source of interest or synthesis from known or predicted sequence information, and may include sequences designed to have desired parameters.

The term “nucleic acid” or “nucleic acid molecule” refers to, e.g., DNA or RNA (e.g., mRNA). The nucleic acid molecules can be double-stranded or single-stranded; single stranded RNA or DNA can be the coding (sense) strand or the non-coding (antisense) strand.

The term “isolated” nucleic acid, such as an isolated protein or nucleic acid as used herein, refers to a biomolecule removed from the context in which the biomolecule exists in nature. An isolated biomolecule can be, in some instances, partially or substantially purified. For example, an isolated nucleic acid molecule can be a nucleic acid sequence that has been excised from the chromosome, genome, or episome into which it is integrated in nature.

A “purified” nucleic acid molecule or nucleotide sequence, or protein or polypeptide sequence, is substantially free of cellular material and cellular components. The purified nucleic acid molecule or protein may be free of chemicals beyond buffer or solvent, for example. “Substantially free” is not intended to mean that other components beyond the novel nucleic acid molecules are undetectable.

“Exogenous nucleic acid molecule” or “exogenous gene” refers to a nucleic acid molecule or gene that has been introduced (“transformed”) into a cell. A transformed cell may be referred to as a recombinant cell, into which additional exogenous gene(s) may be introduced. A descendent of a cell transformed with a nucleic acid molecule is also referred to as “transformed” if it has inherited the exogenous nucleic acid molecule. The exogenous gene may be from a different species (and so “heterologous”), or from the same species (and so “homologous”), relative to the cell being transformed. An “endogenous” nucleic acid molecule, gene or protein is a native nucleic acid molecule, gene or protein as it occurs in, or is naturally produced by, the host.

The term “heterologous” when used in reference to a polynucleotide, a gene, a nucleic acid, a polypeptide, or an enzyme refers to a polynucleotide, gene, a nucleic acid, polypeptide, or an enzyme not derived from the host species. For example, “heterologous gene” or “heterologous nucleic acid sequence” as used herein, refers to a gene or nucleic acid sequence from a different species than the species of the host organism it is introduced into. When referring to a gene regulatory sequence or to an auxiliary nucleic acid sequence used for maintaining or manipulating a gene sequence (e.g. a 5′ untranslated region, 3′ untranslated region, poly A addition sequence, intron sequence, splice site, ribosome binding site, internal ribosome entry sequence, genome homology region, recombination site, etc.) or to a nucleic acid sequence encoding a protein domain or protein localization sequence, “heterologous” means that the regulatory or auxiliary sequence or sequence encoding a protein domain or localization sequence is from a different source than the gene with which the regulatory or auxiliary nucleic acid sequence or nucleic acid sequence encoding a protein domain or localization sequence is juxtaposed in a construct, genome, chromosome or episome. Thus, a promoter operably linked to a gene to which it is not operably linked to in its natural state may be referred to herein as a “heterologous promoter,” even though the promoter may be derived from the same species as the gene to which it is linked. Similarly, when referring to a protein localization sequence or protein domain of an engineered protein, “heterologous” means that the localization sequence or protein domain is derived from a protein different from that into which it is incorporated by genetic engineering.

The term “recombinant” or “engineered” nucleic acid molecule as used herein, refers to a nucleic acid molecule that has been altered through human intervention. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes cojoined nucleotide sequences that are not cojoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

The term “recombinant protein” as used herein refers to a protein produced by genetic engineering. The terms “peptide,” “polypeptide” and “protein” are used interchangeably herein, although “peptide” may be used to refer to a polypeptide having no more than about 100 amino acids, or no more than about 60 amino acids.

When applied to organisms, the terms “transgenic” or “recombinant” or “engineered” or “genetically engineered” refer to organisms that have been manipulated by introduction of an exogenous or recombinant nucleic acid sequence into the organism. Non-limiting examples of such manipulations include gene knockouts, targeted mutations and gene replacement, promoter replacement, deletion, or insertion, as well as introduction of transgenes into the organism. For example, a transgenic microorganism can include an introduced exogenous regulatory sequence, for example a promoter sequence, operably linked to an endogenous gene of the transgenic microorganism. Recombinant or genetically engineered organisms can also be organisms into which constructs for gene “knock down” have been introduced. Such constructs include, but are not limited to, RNAi, microRNA, shRNA, antisense, and ribozyme constructs. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome. As used herein, “recombinant microorganism” or “recombinant host cell” includes progeny or derivatives of the recombinant microorganisms of the invention. Because certain modifications may occur in succeeding generations from either mutation or environmental influences, such progeny or derivatives may not, in fact, be identical to the parent cell, but are still included within the scope of the term as used herein.

The term “expression cassette” as used herein, refers to a nucleic acid construct that encodes a protein or functional RNA (e.g. a tRNA, a short hairpin RNA, one or more microRNAs, a ribosomal RNA, etc.) operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

“Regulatory sequence”, “regulatory element”, or “regulatory element sequence” refers to a nucleotide sequence located upstream (5′), within, or downstream (3′) of a coding sequence or functional RNA-encoding sequence. Transcription of the coding sequence or functional RNA-encoding sequence and/or translation of an RNA molecule resulting from transcription of the coding sequence are typically affected by the presence or absence of the regulatory sequence. These regulatory element sequences may comprise promoters, cis-elements, enhancers, terminators, or introns. Regulatory elements may be isolated or identified from untranslated regions (UTRs) from a particular polynucleotide sequence. Any of the regulatory elements described herein may be present in a chimeric or hybrid regulatory expression element. Any of the regulatory elements described herein may be present in a recombinant construct of the present invention.

The terms “promoter”, “promoter region”, or “promoter sequence” refer to a nucleic acid sequence capable of binding RNA polymerase to initiate transcription of a gene in a 5′ to 3′ (“downstream”) direction. A gene is “under the control of” or “regulated by” a promoter when the binding of RNA polymerase to the promoter is the proximate cause of said gene's transcription. The promoter or promoter region typically provides a recognition site for RNA polymerase and other factors necessary for proper initiation of transcription. A promoter may be isolated from the 5′ untranslated region (5′ UTR) of a genomic copy of a gene. Alternatively, a promoter may be synthetically produced or designed by altering known DNA elements. Also considered are chimeric promoters that combine sequences of one promoter with sequences of another promoter. Promoters may be defined by their expression pattern based on, for example, metabolic, environmental, or developmental conditions. A promoter can be used as a regulatory element for modulating expression of an operably linked transcribable polynucleotide molecule, e.g., a coding sequence or functional RNA sequence. Promoters may contain, in addition to sequences recognized by RNA polymerase and, preferably, other transcription factors, regulatory sequence elements such as cis-elements or enhancer domains that affect the transcription of operably linked genes.

The term “inducible” promoter refers to a promoter having activity dependent on environmental and developmental conditions. The activity of an inducible promoter is dependent on the external environment, such as light and culture medium composition. In some examples, an inducible promoter is inactive in the presence of one or more nutrients (e.g., inorganic phosphates) but active when the one or more nutrients is/are absent. Thus, an inducible promoter is a promoter that is active in response to particular environmental conditions, such as the presence or absence of a nutrient or regulator, the presence or absence of light, etc. In contrast, a “constitutive” promoter is a promoter that is active under most environmental and developmental conditions.

The term “operably linked,” as used herein, denotes a configuration in which a control sequence or localization sequence is placed at an appropriate position relative to a sequence that encodes a polypeptide or functional RNA such that the control sequence directs or regulates the expression or localization of the mRNA encoding the polypeptide, the polypeptide, and/or the functional RNA. Thus, a promoter is in operable linkage with a nucleic acid sequence if it can mediate transcription of the nucleic acid sequence. When introduced into a host cell, an expression cassette that includes a control sequence can result in transcription of the gene to which it is operably linked and/or translation of an encoded RNA or polypeptide under appropriate conditions. Antisense or sense constructs that are not or cannot be translated are not excluded by this definition. In the case of both expression of transgenes and suppression of endogenous genes (e.g., by antisense, or sense suppression) one of ordinary skill will recognize that the inserted polynucleotide sequence need not be identical, but may be only substantially identical to a sequence of the gene from which it was derived. As explained herein, these substantially identical variants are specifically covered by reference to a specific nucleic acid sequence.

The term “selectable marker” or “selectable marker gene” as used herein includes any gene that confers a phenotype on a cell in which it is expressed to facilitate the selection of cells that are transfected or transformed with a nucleic acid construct of the invention. The term may also be used to refer to gene products that effectuate said phenotypes.

A “reporter gene” is a gene encoding a protein that is detectable or has an activity that produces a detectable product. A reporter gene can encode a visual marker or enzyme that produces a detectable signal. Non-limiting examples include: a β-glucuronidase gene, a β-galactosidase gene, or a gene encoding a fluorescent protein, including but not limited to a blue, cyan, green, red, or yellow fluorescent protein, a photoconvertible, photoswitchable, or optical highlighter fluorescent protein, or any of variant thereof, including, without limitation, codon-optimized, rapidly folding, monomeric, increased stability, and enhanced fluorescence variants.

The term “transformation” as used herein refers to the introduction of one or more exogenous nucleic acid sequences or polynucleotides into a host cell or organism by using one or more physical, chemical, or biological methods. Physical and chemical methods of transformation (i.e., “transfection”) include, by way of non-limiting example, polyethylene glycol (PEG) mediated transformation. Biological methods of transformation include, by way of non-limiting example, transfer of DNA using engineered viruses or microbes (e.g., E. coli).

### Isolated Nucleic Acid Molecule

An isolated nucleic acid molecule according to the present disclosure may be a molecule of DNA or a molecule of RNA. It may be single stranded or double stranded. It may optionally be a mRNA. It may optionally have a length between 50 and 5000 bases or, where appropriate, for a double stranded molecule, base pairs. For example, between 100 and 5000 bases/base pairs, for example between 200 and 4000 bases/base pairs, for example between 500 and 5000 bases/base pairs, for example between 1000 and 5000 or 1000 and 2000, or between 200 and 2000 bases/base pairs. It may optionally be chemically modified, for example methylated, acetylated or 5′-capped.

A promoter provided herein can optionally be an inducible promoter (e.g., a promoter active in culture conditions in which one or more specific ingredients are present or deficient, but not in culture conditions in which such conditions do not apply.

A promoter provided herein may optionally be a proximal promoter. A proximal promoter sequence may optionally be approximately 250 basepairs (bp) upstream of the translational start site of the open reading frame of the gene to be transcribed and may contain, in addition to sequences for binding RNA polymerase, specific transcription factor binding sites. Some promoters of aspects of the present disclosure may also include a distal sequence upstream of the gene that may contain additional regulatory elements, often with a weaker influence than the proximal promoter. Further, promoters or portions of promoters provided herein may optionally be combined in series to achieve a stronger level of expression or a more complex pattern of regulation. Particularly preferred promotors include the native pH16 promotor as well as pHyaA, Lac, Trc, BrkB and T7 promotors and also derivatives thereof, such derivatives retaining at least 50% of promotor activity and at least 80% sequence identity with the corresponding non-derivative promotor.

A promoter provided herein can optionally comprise further elements in addition to the elements already described herein. Additional regulatory element sequences contemplated herein for use with the promoters provided herein include cis regulatory elements (e.g., further promoters, enhancers, silencers, operators) and trans regulatory elements. Regulatory elements may be isolated or identified from untranslated regions (UTRs) from a particular polynucleotide sequence. Any of the regulatory elements described herein may optionally be present in a chimeric or hybrid regulatory expression element.

### Expression Cassettes

Expression cassettes are also provided herein. The expression cassettes comprise a promoter, and a gene encoding a polypeptide according to the invention, wherein the gene is operably linked to the promoter. The gene may optionally be positioned downstream of the promoter sequence. Optionally, the expression cassette also comprises one or more additional regulatory elements as described herein. The basic techniques for operably linking two or more sequences of DNA together are familiar to the person of ordinary skill in the art.

### Vectors

Also provided herein are vectors that comprise the expression cassettes provided herein. The vector can be a plasmid. The vector can be a shuttle vector, such as a pURB500 shuttle vector or a pURB500 shuttle vector derivative.

A vector provided herein may optionally further comprise one or more selectable markers and/or reporter genes. For example, in addition to the expression cassette provided herein, the vector may optionally further comprise one or more of: a selectable marker gene, a reporter gene, an origin of replication, and one or more sequences for promoting integration of the expression cassette into the host genome.

By way of example, a vector that includes an expression cassette may optionally include, as one or more selectable markers, one or more genes conferring resistance to an antibiotic or antibiotics (e.g., puromycin, neomycin, 8-azahypoxanthine, 6-azauracil) so that transformants can be selected by exposing the cells to the agent(s) and selecting those cells which survive the encounter. The selectable marker can optionally be operably linked to and/or under the control of a promoter. The promoter regulating expression of the selectable marker may be inducible (e.g. autoinducible) and can be, for example, any promoter provided herein, or another promoter. The selectable marker may optionally be placed under the control of the expression cassette promoter.

If a vector provided herein that includes an expression cassette lacks a selectable marker gene, transformants may be selected by routine methods familiar to those skilled in the art, such as, by way of a non-limiting example, extracting nucleic acid from the putative transformants and screening by PCR.

Alternatively or in addition, transformants may be screened by detecting expression of a reporter gene, such as but not limited to a gene encoding a fluorescent protein, such as any of the blue, cyan, green, red, yellow, photoconvertible, or photoswitchable fluorescent proteins or any of their variants. The reporter gene can be operably linked to and/or under the control of a promoter. The promoter regulating expression of the reporter gene may be inducible and can be, for example, any promoter provided herein, or another promoter. The reporter gene may optionally be placed under the control of the expression cassette promoter.

In addition to the promoters provided herein, one skilled in the art would know various promoters, introns, enhancers, promoter proximal DNA element, initiator element, transit peptides, targeting signal sequences, 5′ and 3′ untranslated regions (UTRs), IRES, 2A sequences, and terminator sequences, as well as other molecules involved in the regulation of gene expression that are useful in the design of effective expression vectors. The expression vector may contain one or more enhancer elements. Enhancers are short regions of DNA that can bind trans-acting factors to enhance transcription levels. Although enhancers usually act in cis, an enhancer need not be particularly close to its target gene. Enhancers can sometimes be located in introns.

The vector can further include one or more additional genes or constructs for transfer into the host cell, which can optionally be operably linked to a promoter provided herein, or can optionally be operably linked to another promoter. Alternatively (or additionally) one or more additional genes or constructs for transfer into the host all can optionally be provided on further vectors or a cell may be chosen that already contains one or more additional genes or constructs necessary for a specific embodiment of the invention. Of particular relevance are genes and constructs for encoding the T1SS channel (for example hylB and hylO) which may be already present in the cell or provided to the cell in a vector of the invention or an additional vector.

### Cells

A prokaryotic host cell is also provided, transformed with an expression vector as provided herein. Thus, a prokaryotic host cell is provided comprising a vector as described herein. A prokaryotic host cell is provided comprising an isolated nucleic acid molecule as described herein. A prokaryotic host cell is provided comprising an expression cassette as described herein.

Any suitable prokaryotic host cell may be used. For example, a bacterial cell, an archaea cell or a prokaryotic algal cell. In certain embodiments the host cell is gram negative bacterial cell for example an E. coli cell. In other embodiments it may be a pseudomonas species, for example P. aeruginosa or P. putida. In other embodiments the host cell may be a halophile, such as slight halophile (suitable for growth in seawater), a moderate halophile or an extreme halophile. The host cell may be a halophilic archaea (for example a member of the Halobacteriaceae family such as Halobacterium spp. or Halococcus spp.). In some embodiments, a suitable cell may be selected which already has high constitutive levels of T1SS channel expression. In other embodiments the cell may be engineered to upregulate levels of endogenious T1SS channel expression. In other embodiments, an expression construct for T1SS channel proteins may be introduced into the cell.

### HlyA Domain

A HlyA domain according to the invention differs from the wild-type HlyA domain of E. coli (SEQ ID NO.: 1)

SEQ ID NO. 1:
LAYGSQGDLNPLINEISKIISAAGSFDVKEERTAAS
LLQLSGNASDFSYGRNSITLTTSA

in that at least the wild-type amino acid residue at position 25 is replaced with the amino acid residue N. The HlyA domain sequence may optionally differ from SEQ ID NO: 1 in one or more additional respects. In certain embodiments the HlyA domain sequence has an at least 60, 70, 80, 85, 90, 95 or 97% identify to SEQ ID NO: 1.

### PhaP1 Domain

According to certain embodiments PhaP1 domain differs from the wildtype Ralstonia eutropha sequence (SEQ ID NO: 3)

SEQ ID NO: 3:
MILTPEQVAAAQKANLETLFGLTTKAFEGVEKLVELNLQVVKTSF
AEGVDNAKKALSAKDAQELLAIQAAAVQPVAEKTLAYTRHLYEIA
SETQSEFTKVAEAQLAEGSKNVQALVENLAKNAPAGSESTVAIVK
SAISAANNAYESVQKATKQAVEIAETNF

in one or more of the following respects:
the presence of amino acid residue N at position 24
the presence of amino acid residues ATL at positions 43 to 45
the presence of amino acid residue N at position 48
the presence of amino acid residue E at position 50
the presence of amino acid residue L at position 67
the presence of amino acid residue L at position 71
the presence of amino acid residue V at position 79
the presence of amino acid residue S at position 83
the presence of amino acid residue A at position 95
the presence of amino acid residue I at position 105
the presence of amino acid residue D at position 117
the presence of amino acid residue V at position 119
the presence of amino acid residue A at position 130
the presence of amino acid residue L at position 133
the presence of amino acid residue V at position 138
the presence of amino acid residue S at position 161.

According to certain embodiments, the PhaP1 domain differs from the wildtype Ralstonia eutropha sequence (SEQ ID NO:3) in two or more of the ways listed above.

According to certain embodiments, the PhaP1 domain differs from the wildtype Ralstonia eutropha sequence (SEQ ID NO:3) in three or more of the ways listed above.

According to certain embodiments, the PhaP1 domain differs from the wildtype Ralstonia eutropha sequence (SEQ ID NO:3) in four or more of the ways listed above.

According to certain embodiments, the PhaP1 domain differs from the wildtype Ralstonia eutropha sequence (SEQ ID NO:3) in five or more of the ways listed above.

According to certain embodiments, the PhaP1 domain differs from the wildtype Ralstonia eutropha sequence (SEQ ID NO:3) in six or more of the ways listed above.

According to certain embodiments, the PhaP1 domain differs from the wildtype Ralstonia eutropha sequence (SEQ ID NO:3) in seven or more of the ways listed above.

According to certain embodiments, the PhaP1 domain differs from the wildtype Ralstonia eutropha sequence (SEQ ID NO:3) in eight or more of the ways listed above.

According to certain embodiments, the PhaP1 domain differs from the wildtype Ralstonia eutropha sequence (SEQ ID NO:3) in nine or more of the ways listed above.

According to certain embodiments, the PhaP1 domain differs from the wildtype Ralstonia eutropha sequence (SEQ ID NO:3) in ten or more of the ways listed above.

According to certain embodiments, the PhaP1 domain differs from the wildtype Ralstonia eutropha sequence (SEQ ID NO:3) in eleven or more of the ways listed above.

According to certain embodiments, the PhaP1 domain differs from the wildtype Ralstonia eutropha sequence (SEQ ID NO:3) in twelve or more of the ways listed above.

According to certain embodiments, the PhaP1 domain differs from the wildtype Ralstonia eutropha sequence (SEQ ID NO:3) in thirteen or more of the ways listed above.

According to certain embodiments, the PhaP1 domain differs from the wildtype Ralstonia eutropha sequence (SEQ ID NO:3) in fourteen or more of the ways listed above.

According to certain embodiments, the PhaP1 domain differs from the wildtype Ralstonia eutropha sequence (SEQ ID NO:3) in fifteen or more of the ways listed above.

According to certain embodiments, the PhaP1 domain differs from the wildtype Ralstonia eutropha sequence (SEQ ID NO:3) in sixteen or more of the ways listed above.

According to certain embodiments, the PhaP1 domain differs from the wildtype Ralstonia eutropha sequence (SEQ ID NO:3) in all of the ways listed above.

The PhaP1 domain sequence may optionally differ from SEQ ID NO: 3 in one or more additional respects. In certain embodiments the PhaP1 domain sequence has an at least 60, 70, 80, 85, 90, 95 or 97% identify to SEQ ID NO: 3.

According to alternative embodiments the PhaP1 domain comprises a sequence of, or derived from the Artic Pseudomonas sp. B1 4-6 genome (PhaP1ₚₛ) given as SEQ ID NO: 4 below:

SEQ ID NO: 4:
MSFFNSEKLQTAQKANLDLLQQISGKVFASVEQLSQLQFKALRDS
TEEHFEGVRKLLAVRDPQGFADLQASFTQPSAQAERLAEFNRQVQ
ALIVGTQSDIAKLTSSQVEAGTQQVNEFVESISKNAPAGSEPVVA
AFKSGLANASTVFESAQKAVKQASDVAQSNFDAATKAAPEASAKA
TSGNK

A PhaP1ₚₛ domain sequence according to the invention may optionally differ from SEQ ID NO: 4 in one or more respects. In certain embodiments a PhaP1ₚₛ domain sequence has an at least 60, 70, 80, 85, 90, 95 or 97% identify to SEQ ID NO: 4. It has been found that use of PhaP1ₚₛ (and sequences showing significant identity therewith) is especially suitable for PHA production using hydrolysate feed-stocks, for example they may be especially suitable for use with lignocellulosic feedstocks.

### Additional Gene Constructs for Improved Expression

In preferred embodiments of the present disclosure, there is also provided phaA, phaB and phaC sequences which have been optimised for PHA production. It will be appreciated that genetic constructs suitable for improved PHA production are especially suitable for use in conjunction with genetic constructs described herein for improved PHA secretion. In this context the term “improved PHA production” is to be understood as encompassing both improvements to the rate or amount of PHA produced and also, alternatively or additionally, to improvements in the quality, type, purity or any other property of the PHA produced. Accordingly, isolated nucleic acid of the first or ninth aspect of the present disclosure may optionally, additionally include phaA, phaB or phaC sequences (preferably all three). However, in certain embodiments the PhaP1-HlyAs sequences and phaA, phaB or phaC sequences may be provided on different nucleic acid molecules. Independently, phaA, phaB or phaC sequences are preferably provided together on a single nucleic acid molecule which may optionally be an isolated nucleic acid molecule according to the first or ninth aspect of the present disclosure.

A plasmid according to the second or tenth aspect of the present disclosure may additionally comprise nucleic acid phaA, phaB or phaC sequences (preferably all three). However, in some embodiments aphaA, phaB or phaC may be provided on a separate plasmid. According to such embodiments a plasmid according to the second or tenth aspect of the invention may be provided in conjunction (i.e. packaged together with or provided together with indications or instructions for conjunctive use with the plasmid according to the invention. According to certain embodiments cells may be optimised for production of preferred PHA co-polymers by modification and/or selection of specific phaA, phaB and/or phaC sequences)

Similar considerations apply to expression cassettes in accordance with the third and eleventh aspect of the present disclosure and to vectors in accordance with the invention, that is to say they may further comprise phaA, phaB or phaC sequences (preferably all three) or phaA, phaB or phaC sequences may optionally be provided on a separate expression vector which may optionally be provided with (for example, packaged with or provided together with indications or instructions for conjunctive use) an expression cassette, or vector according to the invemtion. According to certain embodiments cells may be optimised for production of preferred PHA co-polymers by modification and/or selection of specific phaA, phaB and/or phaC sequences, and/or combinations thereof) an additional expression vector comprising phaA, phaB or phaC sequences.

A prokaryotic cell according to the fifth or thirteenth aspect of the present disclosure or a culture according to the sixth or fourteenth aspect of the present disclosure may comprise a plasmid or expression vector of the present disclosure and optionally phaA, phaB or phaC sequences separately or as part of the plasmid or expression vector of the present disclosure as described above.

Yang et al. (2012) discusses how the properties of PHAs expressed in microbial cells may be improved by the introduction of a propionyl-CoA transferase (pct) gene sequence alongside the PHA biosynthetic gene sequences and also that in place of the trio of PHA biosynthetic genes described above—PhaA, PhaB and PhaC—PHAs, including those with improved mechanical and/or processing properties may be obtained by use of a combination of the following PHA biosynthetic genes—BtkB (β-ketothiolase), PhaB and PhaC. Such combinations of PHA biosynthetic genes are also found to be suitable for use in organisms which have been engineered secrete PHAs with improved efficiency in accordance with the invention by use of a protein having a PhaP1 domain and a HlyA domain which has been mutated in accordance with the invention.

Accordingly, in alternative preferred embodiments the invention provides a BtkB sequence in addition to, or as an alternative to, a PhaA sequence, such that the invention also encompasses embodiments wherein there is also provided BtkB, phaA, phaB and phaC sequences which have been optimised for PHA production, and also embodiments wherein there is also provided BtkB, phaB and phaC sequences (optionally without a PhaA sequence) which have been optimised for PHA production. It will be appreciated that genetic constructs suitable for improved PHA production are especially suitable for use in conjunction with genetic constructs described herein for improved PHA secretion. Accordingly, isolated nucleic acid of the first or ninth aspect of the present disclosure may optionally, additionally include BtkB, phaB and phaC sequences (preferably all three) or BkB, phaA, phaB and phaC sequences (preferably all four). However, in certain embodiments the PhaP1-HlyAs sequences and BtkB, phaB and phaC sequences or BtkB, phaA, phaB and phaC sequences may be provided on different nucleic acid molecules. Independently, BkB, phaB or phaC sequences or BkB, phaA, phaB and phaC sequences are preferably provided together on a single nucleic acid molecule which may optionally be an isolated nucleic acid molecule according to the first or ninth aspect of the present disclosure.

A plasmid according to the second or tenth aspect of the present disclosure may additionally comprise nucleic acid BtkB, phaB and phaC sequences (preferably all three) or BkB, phaA, phaB and phaC sequences (preferably all four). However, in some embodiments one or more of a BtkB, phaA, phaB or phaC sequence may be provided on a separate plasmid. According to such embodiments a plasmid according to the second or tenth aspect of the invention may be provided in conjunction (i.e., packaged together with or provided together with indications or instructions for conjunctive use) with a plasmid of the invention. According to certain embodiments cells may be optimised for production of preferred PHA co-polymers by modification and/or selection of specific BkB, phaA, phaB and/or phaC sequences and/or combinations thereof).

Similar considerations apply to expression cassettes in accordance with the third and eleventh aspect of the present disclosure and to vectors in accordance with the invention, that is to say they may further comprise BtkB, phaA, phaB or phaC sequences (preferably all four) or BkB, phaB or phaC sequences (preferably all three) They may optionally be provided on a separate expression cassette which may optionally be provided with (for example, packaged with or provided together with indications or instructions for conjunctive use) an expression cassette or vector of the invention. According to certain embodiments cells may be optimised for production of preferred PHA co-polymers by modification and/or selection of specific BtkB, phaA, phaB and/or phaC sequences).

A prokaryotic cell according to the fifth or thirteenth aspect of the present disclosure, or a culture according to the sixth or fourteenth aspect of the present disclosure may comprise a plasmid or expression vector of the present disclosure and optionally BtkB, phaA, phaB and/or phaC sequences (for example, BkB, phaA, phaB and phaC sequences, or BkB, phaB and phaC sequences) separately or as part of the plasmid or expression vector of the present disclosure as described above. When provided separately, they may be provided for conjunctive use for example, packaged together or provided together with indications or instructions for conjunctive use.

Accordingly, in alternative preferred optional embodiments the invention provides a pct sequence in addition to the PHA biosynthesis sequences (such as BtkB, phaA, phaB and phaC, or BkB, phaB and phaC) described above, such that the invention also encompasses embodiments wherein there is also provided pct sequences which have been optimised for PHA production, and also embodiments wherein it will be appreciated that genetic constructs suitable for improved PHA production are especially suitable for use in conjunction with genetic constructs described therein for improved PHA secretion. Accordingly, isolated nucleic acid of the first or ninth aspect of the present disclosure may optionally, additionally include pct, BtkB, phaB and phaC sequences (preferably all four) or pct, BtkB, phaA, phaB and phaC sequences (preferably all five). However, in certain embodiments the PhaP1-HlyAs sequences and pct, BtkB, phaB and phaC sequences or pct, BtkB, phaA, phaB and phaC sequences may be provided on different nucleic acid molecules. Independently, pct, BtkB, phaB or phaC sequences or pct, BtkB, phaA, phaB and phaC sequences are preferably provided together on a single nucleic acid molecule which may optionally be an isolated nucleic acid molecule according to the first or ninth aspect of the present disclosure.

A plasmid according to the second or tenth aspect of the present disclosure may additionally comprise nucleic acid pct, BtkB, phaB and phaC sequences (preferably all four) or pct, BtkB, phaA, phaB and phaC sequences (preferably all five). However, in some embodiments one or more of a pct, BtkB, phaA, phaB or phaC sequence may be provided on a separate plasmid. According to such embodiments a plasmid according to the second or tenth aspect of the invention may be provided in conjunction (i.e., packaged together with or provided together with indications or instructions for conjunctive use) with a nucleic acid of the invention. According to certain embodiments cells may be optimised for production of preferred PHA co-polymers by modification and/or selection of specific pct, BtkB, phaA, phaB and/or phaC sequences) a nucleic acid of the invention.

Similar considerations apply to expression cassettes in accordance with the third and eleventh aspect of the present disclosure and to vectors in accordance with the invention, that is to say they may further comprise pct, BtkB, phaA, phaB or phaC sequences (preferably all five) or pct, BtkB, phaB or phaC sequences (preferably all four) They may optionally be provided on a separate expression vector which may optionally be provided with (for example, packaged with or provided together with indications or instructions for conjunctive use. According to certain embodiments cells may be optimised for production of preferred PHA co-polymers by modification and/or selection of specific pct, BkB, phaA, phaB and/or phaC sequences) an expression cassette or vector of the invention.

A prokaryotic cell according to the fifth or thirteenth aspect of the present disclosure or a culture according to the sixth or fourteenth aspect of the present disclosure may comprise a plasmid or expression vector of the present disclosure and optionally pct, BtkB, phaA, phaB and/or phaC sequences (for example, pct, BtkB, phaA, phaB and phaC sequences, or pct, BkB, phaB and phaC sequences) separately or as part of the plasmid or expression vector of the present disclosure as described above.

### Methods of Obtaining PHA

According to a seventh or fourteenth aspect of the present disclosure there is provided a method of obtaining PHA comprising culturing prokaryotic cells, respectively of the fifth or twelfth aspect of the present disclosure for sufficient time and under suitable conditions for said cells to produce PHA and secrete it into the extracellular medium and separating PHA from the extracellular medium.

Culturing may be carried out in any suitable culture vessel. For example, culturing may be carried out in one or more chemostat vessels. Any suitable culture medium may be used which meets the nutritional requirements of the organism. In certain embodiments the culture medium comprises a lignocellulosic biomas stream as a nutritional source. For example, the culture medium may derive from biomas waste or residues from one or more of residues from the pulp/paper residues from the forestry industry (for example timber off cuts and bark). Biomas streams may be preprocessed for example mechanically disrupted before being added to the culture.

A culture according to the present disclosure may be a chemostat continuous culture lasting at least 10, 20, 30, 40 or 50 days. Secreted PHA may be separated from the culture continuously or periodically by sedimentation, filtration, centrifugation or floatation.

### Method for Transforming a Prokaryotic Cell

According to an eighth or sixteenth aspect the present disclosure provides a method for transforming a prokaryotic cell, the method comprising: introducing a vector according, respectively, to the fourth or twelfth aspect of the present disclosure into the prokaryotic cell and selecting for a transformed prokaryotic cell.

The transformed prokaryotic cell may optionally be cultural, optionally under selection in accordance with a method of the seventh aspect of the present disclosure.

### PHAs

According to all aspects, the present disclosure relates to PHAs. Preferred PHAs according to certain embodiments include poly-3-hydroxyvalerate (PHV), poly-4-hydroxybutyrate (P4HB) and polyhydroxyhexanoate (PHH) and the co-polymers poly (3-hydroxybutyrate-co-3-hydroxyvalerate (pHBV), poly 3 hydroxybutyrate-co-hydroxyhexanoate (PHBH).

### Interchangeability of Features Between Aspects

It is understood that specific features, such as specific preferred features, of the invention disclosed herein in the context of one aspect of the present disclosure may also be applicable to other aspects of the present disclosure.

### EXAMPLES

The present disclosure is further described in the following non-limiting examples.

### T1SS Secretion System

The FIGURE illustrates application of the invention to E. coli. The FIGURE shows a cell disgramatically into which the machinery for PHA production has to be introduced in two vectors. One vector has pBHR68 backbone and contains the machinery for PHA production including the three operons from Ralstonia eutropha (also known as Cupriavidus necator) for polyhydroxybutyrate (PHB) production. The phaABC operon allows for the three-step enzymatic reaction converting two acetyl-CoA molecules into acetoacetyl-CoA via a condensation reaction catalyzed by β-ketothiolase (phaA), followed by a stereoselective reduction to (R)-3-hydroxybutryl-CoA catalyzed by the NADPH-dependent acetoacetyl-CoA reductase (phaB) and finally the polymerization to PHB via PHB synthase (phaC). (1990, Anderson and Dawes).

Within a first vector was additionally engineered the signaling peptide of the Type I secretion system (TISS), hemolysin A (HlyA), annealed to the PHA phasin protein (PhaP1) for anchoring of the PHA granule. Site-directed modifications of the PhaP1 and HlyA were made to further improve the native sequence in accordance with the invention. Additionally, the PhaP1 protein sequence was exchanged with an alternative gene taken from the Artic Pseudomonas sp. B14-6 genome (PhaP1_{PS}) to allow for enhanced production and inhibitor tolerance using specific hydrolysate feedstocks (2021, Lee H. S. et al.).

As can be seen, the cell also contains a vector encoding components of the T1SS channel (hlyB and hlyD). PhaA, phaB and phaC co-operate to produce a plain PHA granule which is coated with phasin/hlyA which targets the granule for export via the T1SS channel.

What is claimed is:

1. An isolated nucleic acid molecule comprising a sequence encoding a protein having a PhaP1 domain and a HlyA domain, wherein the HlyA domain differs from the wild-type HlyA domain of E. coli (SEQ ID NO.: 1)
SEQ ID NO. 1:
LAYGSQGDLNPLINEISKIISAAGSFDVKEERTAAS
LLQLSGNASDFSYGRNSITLTTSA
in at least the wild-type amino acid residue S (serine) at position 25 (underlined above) is replaced with the amino acid residue N (asparagine).

2. An isolated nucleic acid molecule according to claim 1 wherein the HylA domain has the sequence of SEQ ID NO.: 2 or a sequence having at least 85% identity thereto:
SEQ ID NO. 2:
LAYGS QGDLN PLINE ISKII SAAGN
FDVKEERTAASLLQLSGNASDFSYGRNSITLTTSA.

3. An isolated nucleic acid molecule according to claim 1, wherein the PhaP1 domain differs from the wild-type Ralstonia eutropha sequence (SEQ ID NO:3)
SEQ ID NO: 3:
MILTPEQVAAAQKANLETLFGLTTKAFEGVEKLVELNLQVVKTSF
AEGVDNAKKALSAKDAQELLAIQAAAVQPVAEKTLAYTRHLYEIA
SETQSEFTKVAEAQLAEGSKNVQALVENLAKNAPAGSESTVAIVK
SAISAANNAYESVQKATKQAVEIAETNF
in at least one or more of the following respects:
the presence of amino acid residue N at position 24
the presence of amino acid residues ATL at positions 43 to 45
the presence of amino acid residue N at position 48
the presence of amino acid residue E at position 50
the presence of amino acid residue L at position 67
the presence of amino acid residue L at position 71
the presence of amino acid residue V at position 79
the presence of amino acid residue S at position 83
the presence of amino acid residue A at position 95
the presence of amino acid residue I at position 105
the presence of amino acid residue D at position 117
the presence of amino acid residue V at position 119
the presence of amino acid residue A at position 130
the presence of amino acid residue L at position 133
the presence of amino acid residue V at position 138
the presence of amino acid residue S at position 161.

4. An isolated nucleic acid molecule according to claim 1, wherein the PhaP1 domain has the sequence of SEQ ID NO. 4 or a sequence having at least 85% identity thereto:
SEQ ID NO. 4:
MSFFNSEKLQTAQKANLDLLQQISGKVFASVEQLSQLQFKALRDS
TEEHFEGVRKLLAVRDPQGFADLQASFTQPSAQAERLAEFNRQVQ
ALIVGTQSDIAKLTSSQVEAGTQQVNEFVESISKNAPAGSEPVVA
AFKSGLANASTVFESAQKAVKQASDVAQSNFDAATKAAPEASAKA
TSGNK.

5. An isolated nucleic acid molecule according to claim 1, which is DNA and which optionally additionally comprises one or more promotors to drive expression of the protein.

6. A plasmid comprising an isolated nucleic acid molecule according to claim 1.

7. An expression cassette comprising an isolated nucleic acid molecule according to claim 1.

8. A vector comprising an expression cassette of claim 7.

9. A prokaryotic cell comprising a plasmid of claim 6.

10. A prokaryotic cell according to claim 8 wherein the prokaryotic cell is an E coli cell.

11. A culture comprising prokaryotic cells of claim 9.

12. A culture of claim 11 to which is a semi-continuous or continuous culture.

13. A method of obtaining PHA comprising culturing prokaryotic cells of claim 9 for sufficient time and under suitable conditions for said cells to produce PHA and secrete it into the extracellular medium and separating PHA from the extracellular medium.

14. A method for transforming a prokaryotic cell, the method comprising:
introducing a vector according to claim 8 into the prokaryotic cell and selecting for a transformed prokaryotic cell.

15. An isolated nucleic acid molecule comprising a sequence encoding a protein having a PhaP1 domain and a HlyA domain, wherein the PhaP1 domain differs from the wild-type Ralstonia eutropha sequence (SEQ ID NO:3)
SEQ ID NO: 3:
MILTPEQVAAAQKANLETLFGLTTKAFEGVEKLVELNLQVVKTSF
AEGVDNAKKALSAKDAQELLAIQAAAVQPVAEKTLAYTRHLYEIA
SETQSEFTKVAEAQLAEGSKNVQALVENLAKNAPAGSESTVAIVK
SAISAANNAYESVQKATKQAVEIAETNF
in at least one or more of the following respects:
the presence of amino acid residue N at position 24
the presence of amino acid residues ATL at positions 43 to 45
the presence of amino acid residue N at position 48
the presence of amino acid residue E at position 50
the presence of amino acid residue L at position 67
the presence of amino acid residue L at position 71
the presence of amino acid residue V at position 79
the presence of amino acid residue S at position 83
the presence of amino acid residue A at position 95
the presence of amino acid residue I at position 105
the presence of amino acid residue D at position 117
the presence of amino acid residue V at position 119
the presence of amino acid residue A at position 130
the presence of amino acid residue L at position 133
the presence of amino acid residue V at position 138
the presence of amino acid residue S at position 161.

16. An isolated nucleic acid molecule according to claim 15, wherein the PhaP1 domain has the sequence of SEQ ID NO. 4 or a sequence having at least 85% identity thereto:
SEQ ID NO. 4:
MSFFNSEKLQTAQKANLDLLQQISGKVFASVEQLSQLQFKALRDS
TEEHFEGVRKLLAVRDPQGFADLQASFTQPSAQAERLAEFNRQVQ
ALIVGTQSDIAKLTSSQVEAGTQQVNEFVESISKNAPAGSEPVVA
AFKSGLANASTVFESAQKAVKQASDVAQSNFDAATKAAPEASAKA
TSGNK.

17. An isolated nucleic acid molecule according to claim 15, wherein the HlyA domain differs from the wild-type HlyA domain of E. coli (SEQ ID NO.: 1)
SEQ ID NO. 1:
LAYGSQGDLNPLINEISKIISAAGSFDVKEERTAAS
LLQLSGNASDFSYGRNSITLTTSA.
in at least the wild-type amino acid residue S (serine) at position 25 (underlined above) is replaced with the amino acid residue N (asparagine).

18. An isolated nucleic acid molecule according to claim 17 wherein the HylA domain has the sequence of SEQ ID NO.: 2 or a sequence having at least 85% identity thereto:
SEQ ID NO. 2:
LAYGS QGDLN PLINE ISKII SAAGN
FDVKEERTAASLLQLSGNASDFSYGRNSITLTTSA.

19. An isolated nucleic acid molecule according to claim 15 which is DNA and which optionally additionally comprises one or more promotors to drive expression of the protein.

20. A plasmid comprising an isolated nucleic acid molecule of claim 15.
